(19) United States
(12) Patent Application Publication
(10) Pub. No.: US 2026/0053551 A1
(43) Pub. Date: Feb. 26, 2026

(54) A CATHETER FOR FORMING A FISTULA

(71) Applicant: TVA MEDICAL, INC., Franklin Lakes, NJ (US)

(72) Inventors: Michael Whelan, Enniscorthy, County Wexford (IE); Jakob Wells, Enniscorthy, County Wexford (IE); John O'Shea, Enniscorthy, County Wexford (IE)

(21) Appl. No.: 19/104,588

(22) PCT Filed: Aug. 16, 2022
(86) PCT No.: PCT/EP2022/072842

§ 371 Date: Feb. 18, 2025

(51) Int. Cl. A61B 18/14; A61B 17/00; A61B 17/32; A61B 17/3207; A61B 18/00; A61B 18/04

(52) U.S. Cl. CPC A61B 18/1492; A61B 17/320725; A61B 18/042; A61B 2017/00783; A61B 2017/00876; A61B 2017/0088; A61B 2017/320004; A61B 2018/00428; A61B 2018/00601; A61B 2018/144; A61B 2018/1475

(57) ABSTRACT

A catheter for forming a fistula between two vessels. The catheter comprises a catheter body having a longitudinal axis, an electrode extending radially from the catheter body for contacting a vessel wall and forming the fistula, and a cutting unit disposed proximally or distally of the electrode for cutting a venous valve.

### TECHNICAL FIELD

The present disclosure relates to a catheter for forming a fistula between two blood vessels, a system of forming a fistula between two vessels and a method of forming a fistula using a catheter system.

### BACKGROUND

Peripheral arterial disease (PAD) can result from the occlusion of arteries in the legs and lower extremities such as the feet. Typically, such occlusion is brought about by atherosclerosis, whereby calcified plaque deposited on the walls of an arterial vessel causes narrowing and blockage of the vessel lumen. Severe cases of arterial blockage in the lower extremities can lead to critical limb ischemia (CLI), a chronic condition characterised by severe pain and slow healing of wounds in the affected extremities due to poor circulation of blood. Left untreated, the subject may suffer loss of limbs because of the need to undergo amputation.

Treatment of such diseased arteries may include angioplasty or atherectomy. However, in some circumstances, these treatments are unsuitable, and an alternative solution is necessary. One such alternative is deep vein arterialisation (DVA), whereby blood flow is routed from the diseased artery to a nearby deep vein in order to supply the extremity with blood. Another possible treatment is an endovascular bypass procedure, whereby blood flow is routed out of the artery and back into the artery by a conduit that circumvents the blockage. That conduit may, for example, be a stent graft placed within an adjacent vein.

The process of routing the blood from the artery to an adjacent vein involves the formation of a fistula, which is a passageway connecting the two vessels.

Further, in order for a deep vein arterialization procedure or an endovascular bypass procedure to be effective, the venous valves that normally hinder retrograde blood flow must be made incompetent.

It is known in the art to perform DVA or endovascular bypass procedures using a catheter for forming a fistula and a separate catheter for valve destruction. However, this approach may be limited, for example, by the complexity of the procedure which necessitates multiple steps involving multiple catheters.

In view of the above, there is a need for an improved catheter which can effectively form a fistula between two vessels and effectively destroy venous valves.

There is further a need in the art for a new catheter which reduces the treatment time and number of steps for a DVA or endovascular bypass procedure and makes the procedures simpler by eliminating the need for multiple catheters for forming a fistula and destroying venous valves.

### SUMMARY

In a first aspect of the present disclosure, there is provided a two catheter for forming a fistula between vessels. The catheter comprises a catheter body having a longitudinal axis, an electrode extending radially from the catheter body for contacting a vessel wall and forming the fistula, and a cutting unit disposed proximally or distally of the electrode for cutting a venous valve.

In some embodiments this may result in an improved catheter which can form a fistula and destroy a venous valve to facilitate effective deep vein arterialization or endovascular bypass procedure.

In some embodiments, this may reduce the treatment time for a DVA or endovascular bypass procedure and makes the procedures simpler by eliminating the need for separate catheters for forming a fistula and destroying venous valves, and reducing the number of steps in the procedure.

Throughout this disclosure, the terms “proximal” and “distal” refer to the proximal and distal directions in relation to the catheter, unless otherwise specified. In that case, “proximal” refers to a point on the catheter which is intended to be closer to a physician when the catheter is in use, while “distal” refers to a point on the catheter which is intended to be further away from a physician when the catheter is in use.

Throughout this disclosure, the term ‘fistula’ is used to denote a connection or passageway.

The cutting unit may be expandable.

The cutting unit may have a radially contracted configuration and a radially expanded configuration.

In some embodiments, this may allow the profile of the catheter to be reduced for easier movement through a vessel.

Throughout this description, the ‘radially expanded configuration’ of an element refers to a configuration where the element extends radially further from the catheter body than in the ‘radially contracted configuration’.

The cutting unit may have a plurality of cutting tools.

The cutting tools may be arranged circumferentially around the catheter body.

In some embodiments, this may result in more effective valve destruction.

Each of the cutting tools may extend radially relative to the catheter body.

In some embodiments, this may result in more effective valve destruction.

Each of the cutting tools may have a convex shape relative to the catheter body.

In some embodiments, this may result in more effective valve destruction while reducing or preventing damage to the vessel wall.

Each of the cutting tools may comprise a wire.

The cutting unit may be made of nitinol.

In some embodiments, this may result in better valve destruction.

The cutting tools may extend longitudinally along the catheter body.

Each of the cutting tools may have a proximal end and a distal end.

The proximal end and distal end of each of the cutting tools may be connected to the catheter body.

At least one cutting edge may be disposed between the proximal and distal end of each cutting tool.

In some embodiments, this may result in more effective valve destruction.

Each of the cutting tools may further comprise a tooth positioned adjacent the cutting edge.

In some embodiments, this may result in more effective valve destruction.

Throughout this disclosure, the term ‘abrasive surface’ is used to denote a roughened surface that can scrape away the tissue of a venous valve through friction.

The at least one cutting edge may be a straight edge or a serrated edge.

The cutting edge may be positioned in a recessed portion of the cutting tool.

In some embodiments, this may minimise damage to the vessel wall.

Each of the cutting tools may have a proximal section and a distal section.

The cutting unit may be disposed proximally of the electrode.

The at least one cutting edge may be disposed in the proximal section of each cutting tool.

The at least one cutting edge may be facing proximally.

In some embodiments, this may allow a valve to be destroyed by pulling the first catheter distally.

The cutting unit may be disposed distally of the electrode.

The at least one cutting edge may be disposed in the distal section of each cutting tool.

The at least one cutting edge may be facing distally.

In some embodiments this may allow a valve to be destroyed by pushing the first catheter distally.

The distal end of each cutting tool may be fixed to the catheter body and the proximal end of each cutting tool may be moveable to move the cutting unit between the radially contracted configuration and radially expanded configuration.

The electrode may comprise a distal portion, a proximal portion and an intermediate portion.

The catheter body may comprise a housing with at least one opening.

The intermediate portion of the electrode may extend radially from the opening in the housing of the catheter body.

The housing may be at least partly made from a ceramic material.

In some embodiments, this may allow the housing to better withstand the heat and plasma generated by the electrode.

The electrode may have a radially contracted configuration and a radially expanded configuration.

In some embodiments, this may allow the profile of the catheter to be reduced for easier movement through a vessel.

The distal portion of the electrode may be moveable inside the housing for moving the electrode between the radially contracted configuration and the radially expanded configuration.

In some embodiments, this may provide an effective way to move the electrode between the radially contracted configuration and the radially expanded configuration.

The distal portion of the electrode may be fixed inside the housing and the proximal portion of the electrode may be moveable for moving the electrode between the radially contracted and expanded configurations.

In some embodiments, this may provide an effective way to move the electrode between the radially contracted configuration and the radially expanded configuration. In some embodiments, this may further allow the expansion of the electrode to be controlled.

The electrode may be a ribbon wire.

In some embodiments, this may result in more effective fistula formation.

The electrode may be a leaf spring.

In some embodiments, this may allow the electrode to be bent and flexed without breaking.

In some embodiments, this may allow the electrode to more easily move between the radially contracted configuration and the radially expanded configuration.

Throughout this disclosure, the term ‘leaf spring’ is used to refer to a flexible curved strip of material which can be bent but will regain its original shape when released.

The electrode may have a convex shape relative to the catheter body.

In some embodiments, this may result in better fistula formation.

In a second aspect of the present disclosure, there is provided a system for forming a fistula between two vessels. The system comprises a first catheter according to any of the preceding clauses.

The system may further comprise a second catheter comprising a second housing and a backstop for the electrode.

The backstop may be a recessed backstop which has a portion shaped complementary to the electrode.

In some embodiments, this may allow the electrode to better engage with the backstop and result in better fistula formation.

The backstop may have a concave portion.

The first catheter and the second catheter may each comprise one or more magnets positioned to align the electrode with the backstop.

In some embodiments, this may provide a simpler way to allow exact alignment of the electrode and the backstop.

The system may further comprise a radiofrequency generator for supplying radiofrequency power to the electrode.

The system may further comprise a sheath that is disposed over at least a portion of the catheter body and is slidably moveable along the longitudinal axis of the catheter body.

The sheath may be slidable to move the cutting unit between a radially contracted configuration and a radially expanded configuration.

The sheath may be slidable to move the electrode between a radially contracted configuration and radially expanded configuration.

In some embodiments, this may reduce the number of catheter components and simplifies the mechanism of controlling the movement of both the cutting unit and the electrode between their radially contracted and expanded configuration.

The system may further comprise a handle disposed at a proximal end of the first catheter.

The handle may comprise a sheath sliding mechanism.

The sheath sliding mechanism may comprise a slider for moving the sheath in a proximal or distal direction to unsheathe or sheathe the cutting unit and to thereby change the configuration of the cutting unit between the radially contracted and radially expanded configurations.

In some embodiments, this may provide a simpler way to independently control movement of the cutting unit between the radially contracted configuration and radially expanded configuration.

The sheath sliding mechanism may comprise a slider for moving the sheath in a proximal or distal direction to unsheathe or sheathe the electrode and to thereby change the configuration of the electrode between the radially contracted configuration and radially expanded configuration.

In some embodiments, this may provide a simpler way to independently control movement of the electrode between the radially contracted and radially expanded configurations.

In some embodiments, this may provide a simpler way to control both the movement of the electrode and the cutting unit between the radially contracted and radially expanded configurations.

The handle may further comprise a cutting unit control mechanism for controlling the extent of radial expansion of the cutting unit.

In some embodiments, this allow better control of the expansion of the cutting unit.

The cutting unit control mechanism may comprise a push wire that is connected to the proximal end of the cutting tools.

The handle may further comprise an electrode expansion mechanism for controlling the extent of radial expansion of the electrode.

In some embodiments, this may allow better control of the expansion of the electrode.

The electrode expansion mechanism may comprise a push wire that is connected to a proximal end of the electrode.

According to a third aspect of the present disclosure, there is provided a method of forming a fistula using a catheter having a catheter body with an electrode and a cutting unit disposed proximally or distally of the electrode. The method comprises inserting the catheter into a vein through an access site; moving the electrode from a radially contracted configuration to a radially expanded configuration; forming the fistula by supplying RF energy to the electrode; moving the cutting unit from a radially contracted configuration to a radially expanded configuration; and cutting a valve by moving the catheter longitudinally in a proximal or distal direction.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1 shows a catheter system for forming a fistula according to one of more embodiments shown and described herein.

FIG. 2 shows a cutting unit of the catheter system of FIG. 1 with cutting edges disposed proximally on the cutting tools, according to one or more embodiments shown and described herein.

FIG. 3A shows a cross-sectional side view of the catheter system of FIG. 1 being deployed in a blood vessel system prior to forming a fistula, according to one or more embodiments shown and described herein.

FIG. 3B shows a cross-sectional side view of the catheter system of FIG. 1 disposed at the treatment site for forming a fistula, according to one or more embodiments shown and described herein.

FIG. 3C shows a cross-sectional side view of the catheter system of FIG. 1 during the valve destruction process, according to one or more embodiments shown and described herein.

FIG. 3D shows a cross-sectional side view of a blood vessel system following completion of fistula formation and venous valve destruction, according to one or more embodiments shown and described herein.

FIG. 4 shows an alternative embodiment of a first catheter, according to one or more embodiments shown and described herein.

FIG. 5 shows an alternative embodiment of a cutting unit of the first catheter of FIG. 4 having cutting edges disposed distally on the cutting tools, according to one or more embodiments shown and described herein.

FIG. 6A shows a cross-sectional side view of the catheter of FIG. 4 and a second catheter being deployed in a blood vessel system prior to forming a fistula, according to one or more embodiments shown and described herein.

FIG. 6B shows a cross-sectional side view of the catheter of FIG. 4 and a second catheter during the valve destruction process, according to one or more embodiments shown and described herein.

FIG. 6C shows a cross-sectional side view of the catheter of FIG. 4 and a second catheter disposed at the treatment site for forming a fistula, according to one or more embodiments shown and described herein.

### DETAILED DESCRIPTION

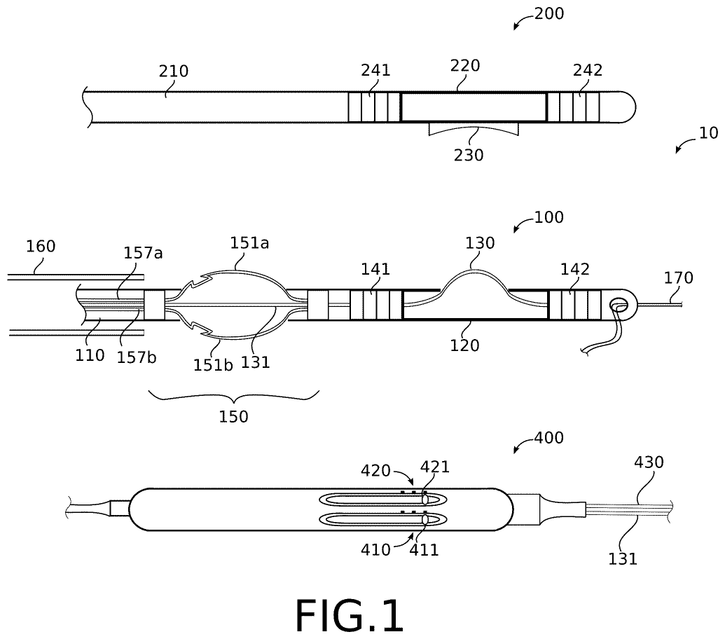

FIG. 1 generally depicts a catheter system 10 for forming a fistula. The system may generally include a first catheter 100 and a second catheter 200 which can be used together to form a fistula between two vessels. The system may further comprise a sheath 160 which may be disposed over at least a portion of the catheter body 110 of catheter 100. The system 10 may further comprise a handle 400 disposed at a proximal end of the first catheter 100.

The first catheter 100 include a catheter body 110. A housing 120 may be disposed near the distal end of the catheter body 110. An electrode 130 may be at least partially disposed in the housing 120 and may extend out of the housing 120 through a first opening in the housing 120. The electrode 130 may have a convex shape relative to the catheter body. The electrode 130 may further have a radially contracted configuration and a radially expanded configuration. FIG. 1 shows the electrode 130 in the radially expanded configuration, where the electrode 130 extends radially further from the housing than in the radially contracted configuration. In the radially contracted configuration, the electrode 130 may be fully disposed within the housing 120 or it may extend from the housing 120 by a small distance. An electrode connecting element 131 may be connected to a proximal end of the electrode 130 and may extend along the length of the catheter body 110 of the first catheter 100. The electrode connecting element 131 may be connected to a source of RF energy, for example in the form of an ESU pencil or RF generator, via the handle 400. The electrode connecting element 131 may be conductive to allow RF energy to be supplied to the electrode 130 via the electrode connecting element 131.

The electrode 130 may be in the form of a ribbon wire and may be made from a number of suitable materials, such as one or more refractory metals. For example, the electrode 130 may comprise tungsten, molybdenum, niobium, tantalum, rhenium, or combinations and alloys thereof. The housing 120 may be made from a non-conductive ceramic material which can withstand the heat and plasma generated by the electrode 130.

The catheter 100 further comprises a cutting unit 150 which may be disposed proximally of the electrode. The cutting unit 150 may comprise a plurality of cutting tools. FIG. 1 shows the cutting unit 150 comprising two cutting tools 151a and 151b, however, the cutting unit 150 may comprise any suitable number of cutting tools, for example, in the range of 2 to 10 cutting tools, such as in the range of 3 to 6 cutting tools. The cutting tools 151a, 151b may be arranged circumferentially around the catheter body 110 and may extend radially from the catheter body 110. Each of the cutting tools 151a, 151b may have a substantially convex shape relative to the catheter body 110 and may extend longitudinally along the catheter body 110.

Similar to the electrode 130, the cutting unit 150 may have a radially contracted configuration and a radially expanded configuration. FIG. 1 shows the cutting unit 150 in the radially expanded configuration, where the cutting unit 150 extends radially further from the catheter body 110 than in the radially contracted configuration. In the radially contracted configuration, the cutting unit 150 may be fully disposed within the catheter body 110 or it may extend from the catheter body 110 by a small distance. A first connecting element 157a may be connected to a proximal end of the cutting tool 151a and may extend along the length of the catheter body 110 of the first catheter 100, while a second connecting element 157b may be connected to a proximal end of the cutting tool 151b and may extend along the length of the catheter body 110 of the first catheter 100. In an alternative embodiment, a single connecting element may be connected to the proximal end of both cutting tools 151a and 151b. Each of the connecting elements 157a, 157b may be in the form of a wire.

The cutting unit 150 may be made from the same materials as the electrode 130 or it may be made, for example, from nitinol. The connecting elements 157a, 157b may be made from the same materials as the cutting unit 150 or may be made, for example, from nitinol.

The first catheter 100 may further comprise a proximal set of magnets 141 and a distal set of magnets 142 which are disposed proximally and distally of the housing 120, respectively.

The second catheter 200 also comprises a catheter body 210 and a second housing 220 having a backstop 230 disposed near the distal end of the catheter body 210. The backstop 230 may have a concave portion which is shaped complimentary to the convex shape of the electrode 130. The second catheter 200 and backstop 230 may also be made from a ceramic material to withstand the heat and plasma generated by the electrode 130. The second catheter 200 may also comprise a proximal set of magnets 241, disposed proximally of the housing 220, and a distal set of magnets 242, disposed distally of the housing 220.

The sheath 160 may be slidably moveable along the longitudinal axis of the catheter body 110 of the first catheter 100. Sliding the sheath 160 along the catheter body 110 in a distal direction to be disposed over the electrode 130 can move the electrode 130 from the radially expanded configuration to the radially contracted configuration. Sliding the sheath 160 along the catheter body 110 away from the electrode 130 in a proximal direction allows the electrode 130 to be moved from the radially contracted configuration to a radially expanded configuration.

Similarly, sliding the sheath 160 distally along the catheter body 110 to be disposed over the cutting unit 150 can move the cutting unit 150 from the radially expanded configuration to the radially contracted configuration. Sliding the sheath 160 proximally along the catheter body 160 away from the cutting unit 150 allows the cutting unit 150 to be moved from the radially contracted configuration to the radially expanded configuration.

The handle 400 may comprise a sheath sliding mechanism (not shown) comprising a slider which allows the sheath 160 to be slidably moved along the longitudinal axis of the catheter body 110 in a proximal and distal direction, as described above.

The handle 400 may further comprise an electrode expansion mechanism 410 for controlling the extent of radial expansion of the electrode 130. The connecting element 131 of the first catheter 100 may be in the form of a push wire. The electrode expansion mechanism 410 may further comprise a slider 411, which is connected to the proximal end of the connecting element 131, that the user can slidably move to pull or push the connecting element 131. In order for the electrode 130 to be moveable between the contracted and expanded configurations, the electrode 130 may have a distal portion that is fixed within the housing 120 and a proximal portion that is moveable within the housing 120. A user can therefore push the connecting element 131 in a distal direction to move the electrode 130 from a radially contracted configuration to a radially expanded configuration. Contrastingly, in order to move the electrode from a radially expanded configuration to a radially contracted configuration, a user can pull the connecting element 131 in a proximal direction.

The connecting element 131 may be made from a number of conductive materials, such as copper, iron or aluminium, for example, or may be made from the same material as the electrode 130. The connecting element 131 may further comprise an insulating coating made from polyimide, for example.

The handle 400 may further comprise a cutting unit control mechanism 420 for controlling the extent of radial expansion of the cutting unit 150. The cutting unit control mechanism 420 may be connected to a push wire 430. A proximal end of the first connecting element 157a and a proximal end of the second connecting element 157b of catheter 100 may be connected to a distal end of a push wire 430. In some embodiments, the cutting unit control mechanism 420 may comprise a slider 421, which is connected to the proximal end of the push wire 430, that the user can slidably move to pull or push the push wire 430. In order for the cutting unit 150 to be moveable between the contracted and expanded configurations, each of the cutting tools 151a and 151b may have a distal end that is fixed to the catheter body 110 and a proximal end that is moveable within the catheter body 110. A user can therefore push the push wire 430 in a distal direction to move the cutting unit 150 from a radially contracted configuration to a radially expanded configuration. Contrastingly, in order to move the cutting unit 150 from a radially expanded configuration to a radially contracted configuration, a user can pull the push wire 430 in a proximal direction.

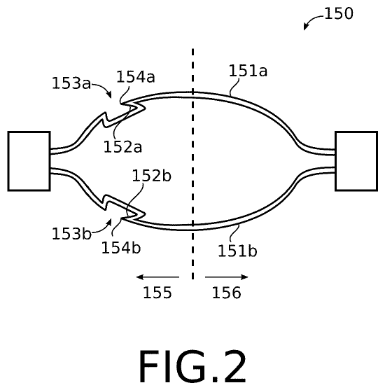

FIG. 2 shows a side view of a cutting unit 150, of the catheter 100 shown in FIG. 1. FIG. 2 is shown as having two cutting tools 151a and 151b. Each of the cutting tools 151a, 151b may comprise at least one cutting edge 152a, 152b disposed between a proximal end and distal end of the cutting tool 151a, 151b. The cutting edges 152a, 152b may be serrated or non-serrated. For example, in FIG. 2, cutting tool 151a has a non-serrated cutting edge 152a and cutting tool 151b has a non-serrated cutting edge 152b. Cutting edges 152a and 152b are positioned in the proximal portion 155 of the cutting unit 150 and are facing in a proximal direction. The cutting edges 152a and 152b may come into contact with a valve when the cutting unit 150 is in the radially expanded configuration and when the catheter 100 is moved in a distal direction through a vein. The cutting edges can destroy the tissue of the valve, for example by ripping or cutting, when moved into contact with the valve. Each of the cutting edges 152a, 152b may be positioned in a recessed portion 153a, 153b of the cutting tool 151a, 151b. For example, in FIG. 2, cutting edge 152a is positioned in recessed portion 153a of cutting tool 151a and cutting edge 152b is positioned in recessed portion 153b of the cutting tool 151b. By positioning the cutting edges 152a, 152b in a recessed portion 153a, 153b of the cutting tools 151a, 151b, this may minimise the contact between the cutting edges 152a, 152b and the vessel wall. Therefore, the cutting unit 150 may provide for effective valve destruction whilst also preventing or minimising damage to the vessel wall. The convex shape of the cutting tools 151a and 151b is also advantageous for minimising damage to the vessel wall as it allows the valve to be more accurately targeted by the cutting edges 152a and 152b. Cutting tools 151a and 151b may further comprise spikes 154a and 154b respectively. In FIG. 2, spikes 154a and 154b are positioned adjacent to their associated cutting edges and face in the same proximal direction as the cutting edges. These spikes 154a, 154b may pierce or engage the valve tissue and thereby allow the cutting edge to better cut the valve tissue.

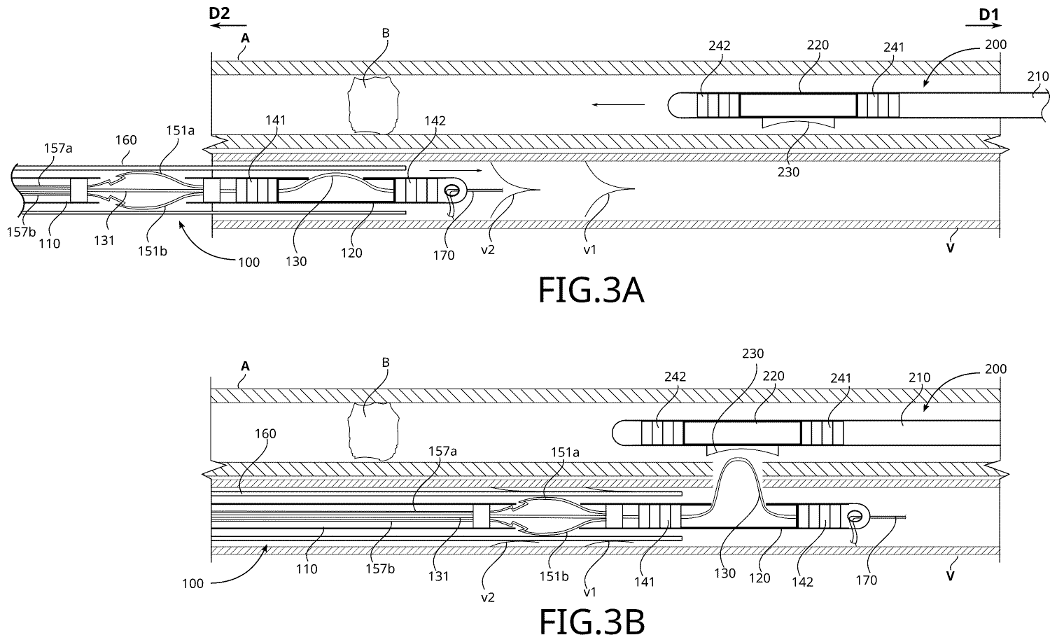

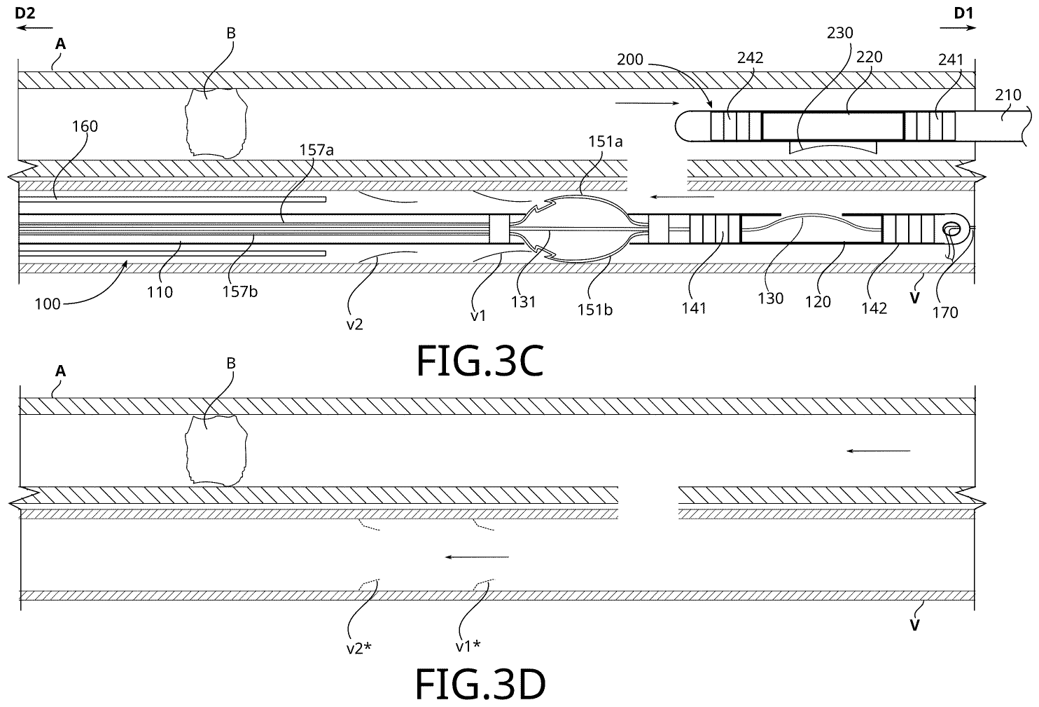

FIGS. 3A-D illustrate a method of forming a fistula between an artery A and vein V and destruction of valves in the vein V in order to circumvent a blockage B in the artery A using catheter system 10. For the method described in FIGS. 3A-D, the direction D1 is a proximal direction in relation to the body of the subject, not the catheter. The direction D2 is a distal direction in relation to the body of the subject, not the catheter.

As shown in FIG. 3A, firstly, the first catheter 100 is introduced into a vein V through an access site and the second catheter 200 is introduced into an artery A through a second access site. The first access site may be positioned distally in direction D2 of the blockage B, while the second access site may be positioned proximally in direction D1 of the blockage B.

The first catheter 100 is then advanced through the vein V in a proximal direction D1 toward a treatment site where the fistula is to be formed. The treatment site may be positioned proximally in direction D1 of the blockage B. The first catheter 100 may be introduced and advanced to the treatment site along a guidewire 170. Similarly, the second catheter 200 is advanced through the artery A in a distal direction D2 toward the treatment site where the fistula is to be formed. The second catheter 200 may also be advanced to the treatment site where the fistula is to be formed along a guidewire (not shown).

The first catheter 100 may be advanced to the treatment site inside sheath 160. The electrode 130 and the cutting unit 150 the radially contracted are shown in FIG. 3A in configuration, with the sheath 160 disposed over the electrode 130 and the cutting unit 150. This reduces the profile of the first catheter 100 and allows the first catheter 100 to be more easily introduced and advanced through the vein V. The second catheter 200 may also be advanced to the treatment site inside a sheath (not shown).

Once the first catheter 100 and the second catheter 200 are positioned at the treatment site, as shown in FIG. 3B, the proximal set of magnets 141 of the first catheter 100 will be attracted to the distal set of magnets 242 of the second catheter 200 and align themselves with each other. Similarly, the distal set of magnets 142 of the first catheter 100 will be attracted to the proximal set of magnets 241 of the second catheter 200 and these sets of magnets will align with each other. This will result in the electrode 130 becoming aligned with the concave backstop 230. The backstop 230 is configured to compress the vessel walls in a localised region for ablation by the electrode 130 of the first catheter 100. The attraction of the magnets also causes the first catheter 100 and second catheter 200 to be pulled closer together, thereby compressing the vessel walls of the artery A and vein V, resulting in more effective fistula formation.

The electrode 130 may then be moved by a user from the radially contracted configuration to the radially expanded configuration, as shown in FIG. 3B. This may be done by pulling back the sheath 160 to allow the electrode 130 to expand from the radially contracted configuration to the radially expanded configuration. A user may further push the electrode connecting element 131 in a distal direction D2 to move the electrode from the radially contracted configuration to the radially expanded configuration or to increase the extent of radial expansion of the electrode in the radially expanded configuration.

In the radially expanded configuration, the electrode 130 has an increased electrode height, which may allow the electrode 130 to more effectively cut through the vessel walls to form a fistula.

A radiofrequency (RF) current may then be supplied to the electrode 130 which causes the electrode 130 to heat up and generate a plasma. The plasma causes rapid dissociation of the molecular bonds in the organic compounds and allows the electrode 130 to cut through the venous and arterial vessel walls until it hits the backstop 230 to form a fistula.

During the fistula formation process, the cutting unit 150 may be sheathed by the sheath 160 and therefore in the radially contracted configuration.

Once the fistula is formed, the electrode 130 may be moved from its radially expanded configuration to its radially contracted configuration, for example using slider 411, as shown in FIG. 3C. This allows the catheter 100 to be more easily moved along the vein V after fistula formation due to the lower profile of the electrode 130.

Once the electrode 130 is returned to its radially contracted configuration, the first catheter 100 may be moved along the vein V such that the cutting unit 150 is positioned proximal to a valve to be destroyed, e.g. valve v1. The first catheter 100 may also be moved along the vein with the electrode 130 in the radially expanded configuration. The cutting unit 150 may then be deployed in the radially expanded configuration, as shown in FIG. 3C. This may be done by pulling the sheath 160 away from the cutting unit 150. A user may further push the push wire 430 that is connected to both the proximal end of the first connecting element 157a and to the proximal end of the second connecting element 157b distally to move the cutting tools 151a and 151b respectively from a radially contracted configuration to a radially expanded configuration or to increase the extent of radial expansion of the cutting tools 151a and 151b in the radially expanded configuration.

After deployment of the cutting unit 150, the catheter 100 may be moved in a distal direction D2 to bring the cutting edges 152a and 152b of the cutting tools 151a and 151b (as shown in FIG. 2) into contact with the valve to be destroyed, e.g. valve v1. The spikes 154a, 154b may engage or pierce the valve tissue. The first catheter 100 is then further pulled in a distal direction D2 such that the cutting edges 152a, 152b cut or rip the valve tissue. The valve destruction process may be supplemented by short longitudinal movements of the first catheter 100 to improve the effectiveness of valve destruction. The first venous valve v1 is thereby rendered incompetent such that it can no longer prevent retrograde blood flow in the vein V. This process of valve destruction can be repeated to destroy a second venous valve v2 and any number of valves as necessary for the treatment procedure.

FIG. 3D shows a cross-sectional side view of a blood vessel system following completion of the fistula formation and venous valve destruction processes according to FIGS. 3B-3C.

The arrows in FIG. 3D depict the flow of blood from the artery A through the fistula and into the vein V, and then through the incompetent valves v1* and v2* in the distal direction. In this case, the vein V is successfully arterialised after deployment of the catheter system 10. The blood flow that is initially blocked by the blockage B in the artery A is re-routed to the vein V and can effectively flow in a retrograde direction in the vein V without hindrance by the venous valves v1, v2.

When performing a deep vein arterialization (DVA) procedure, a stent may be placed within the fistula to stabilise the fistula. When performing an endovascular bypass procedure, a second fistula may be formed distally of the blockage B in a similar manner as explained with respect to FIG. 3B above. A stent graft may then be placed through the first and second fistulas via the vein V, such that the blood flow can circumvent the blockage B.

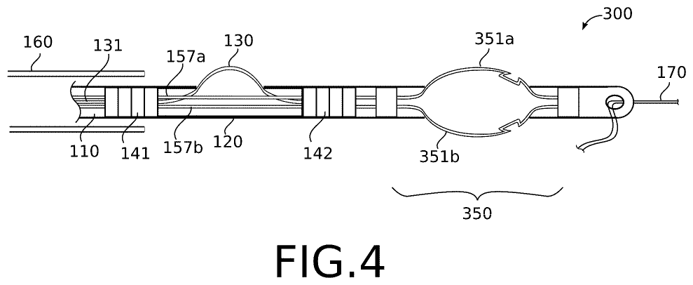

FIG. 4 shows an alternative embodiment of a first catheter 300. Throughout the description, the same reference numerals are used to denote features which are identical across different embodiments.

The first catheter 300 differs from the first catheter 100 in that the cutting unit 350 is disposed distally of the electrode 130.

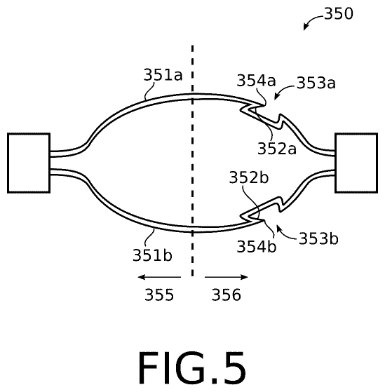

FIG. 5 shows the cutting unit 350 used in the catheter 300 of FIG. 4. Cutting unit 350 is similar to cutting unit 150 of FIG. 2. However, in cutting unit 350, the cutting edges 352a and 352b of cutting tools 351a and 351b are disposed in the distal portion 356 of the cutting unit 350. The cutting edges 352a and 352b and the spikes 354a, 354b are facing distally.

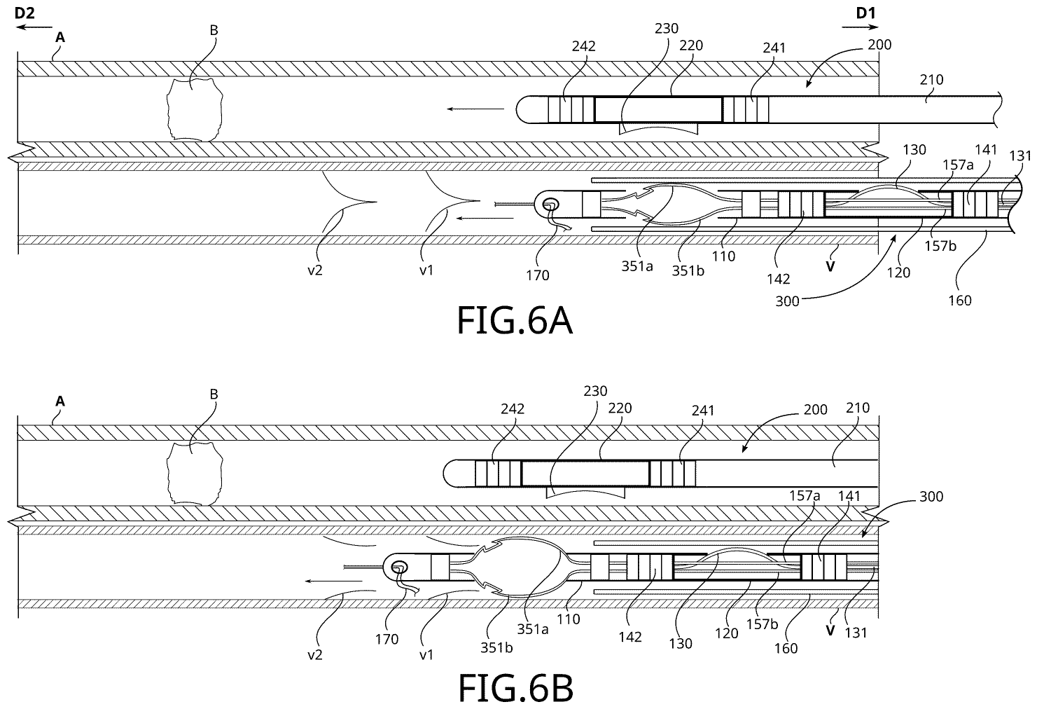

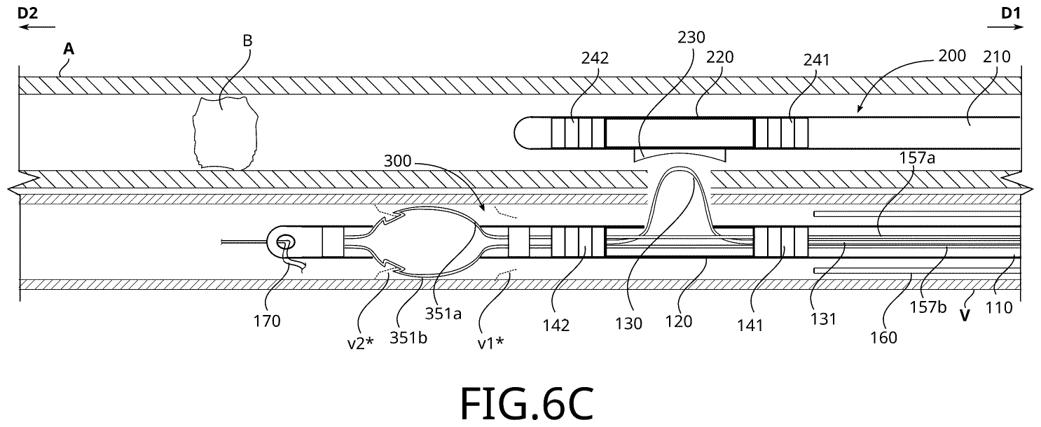

FIGS. 6A-C illustrate a method of forming a fistula between an artery A and vein V and destruction of valves in the vein V in order to circumvent a blockage B in the artery A using catheter system 10, specifically using catheter 300 as the catheter for fistula formation and valve destruction. For the method described in FIGS. 6A-6C, the direction D1 is a proximal direction in relation to the body of the subject, not the catheter. The direction D2 is a distal direction in relation to the body of the subject, not the catheter.

As shown in FIG. 6A, firstly, the first catheter 300 is introduced into a vein V through a first access site and the second catheter 200 is introduced into an artery A through a second access site. In this case, the first and second access sites may be positioned proximally of the blockage B.

The first catheter 300 is then advanced through the vein V in a distal direction D2 toward the treatment site where the fistula is to be formed. The first catheter 300 may be introduced and advanced to the treatment site along a guidewire 170. Similarly, the second catheter 200 is advanced through the artery A in a distal direction D2 toward the treatment site where the fistula is to be formed. The second catheter 200 may also be advanced to the site where the fistula is to be formed along a guidewire (not shown). The first catheter 300 may be advanced to the valve destruction site inside sheath 160, and the second catheter may also be advanced to the fistula formation site inside a sheath (not shown).

The electrode 130 and the cutting unit 150 are shown in FIG. 6A in the radially contracted configuration, with the sheath 160 disposed over the electrode 130 and the cutting unit 150. This reduces the profile of the first catheter 300 and allows the first catheter 300 to be more easily introduced and advanced through the vein V.

Once the first catheter 300 is positioned adjacent to valve is to be destroyed, e.g. proximally adjacent to the valve v1, the cutting unit 150 may then be deployed in its radially expanded configuration, as shown in FIG. 6B. This may be done by slidably moving the sheath 160 away from the cutting unit 150. In some embodiments, after the sheath 160 is slidably moved away from the cutting unit 150, a user can push a push wire 430 that is connected to both the proximal end of the first connecting element 157a and to the proximal end of second connecting element 157b in a distal direction to move the cutting tools 351a and 351b respectively from the radially contracted configuration to the radially expanded configuration or to increase the extent of radial expansion of the cutting tools 351a and 351b in the radially expanded configuration.

After deployment of the cutting unit 150, the first catheter 300 may be moved in a distal direction D2 to bring the distally facing cutting edges 352a and 352b of the cutting tools 351a and 351b (as shown in FIG. 5) into contact with the valve, e.g. v1, to be destroyed. The cutting edges result in damage to the valve tissue for example by ripping, scraping or cutting. In some embodiments, the spikes 354a and 354b also contribute to valve destruction. The valve destruction process may be supplemented by short longitudinal movements of the catheter 300 to improve the effectiveness of valve destruction. The first venous valve v1 is thereby rendered incompetent such that it can no longer prevent retrograde blood flow in the vein V. This process of valve destruction can be repeated to destroy a second venous valve v2 and any number of valves as necessary for the treatment procedure.

Once the valves, e.g. v1 and v2, have been destroyed, the catheter 300 may be moved along the vessel so that the electrode 330 is positioned at the treatment site where the fistula is to be formed. The second catheter 200 may already be positioned at the fistula formation site.

Once the first catheter 300 and the second catheter 200 are positioned at the treatment site, as shown in FIG. 6C, the proximal set of magnets 141 of the catheter will be attracted to the proximal set of magnets 241 of the second catheter and align themselves with each other. Similarly, the distal set of magnets 142 of the catheter 300 will be attracted to the distal set of magnets 242 of the second catheter 200 and these sets of magnets will align with each other. This will result in the electrode 130 becoming aligned with the backstop 130. The sets of magnets may also have the effect of pulling the artery A and vein V closer together.

The electrode 130 may then be moved by a user from the radially contracted configuration to the radially expanded configuration, as shown in FIG. 6C. This may be done by slidably moving the sheath 160 away from the electrode 130. In some embodiments, after the sheath 160 is slidably moved away from the electrode 130, a user can push the proximal end of electrode connecting element 131 to move the electrode from a radially contracted configuration to a radially expanded configuration or to increase the extent of radial expansion of the electrode in the radially expanded configuration.

In the radially expanded configuration, the electrode 130 has an increased electrode height, which allows the electrode 130 to more effectively cut through the vessel walls to form a fistula.

A radiofrequency (RF) current may then be supplied to the electrode 130 which causes the electrode 130 to heat up and generate a plasma. The plasma causes rapid dissociation of the molecular bonds in the organic compounds and allows the electrode 130 to cut through the venous and arterial vessel walls until it hits the backstop 230 to form a fistula.

During the fistula formation process, the cutting unit 350 may remain in the radially expanded configuration without risk of damaging the vessel wall because the cutting edges 352a and 352b of the cutting tools 351a and 351b are positioned to be atraumatic to the vessel wall.

Once the fistula is formed, the electrode 130 may be moved from its radially expanded configuration to its radially contracted configuration. This may be done by slidably moving the sheath 160 to be disposed over the electrode 130 and the cutting unit 350. In some embodiments, a user can pull the connecting element 131 in a proximal direction D1 to move the electrode 130 from a radially expanded configuration to a radially contracted configuration. This allows the catheter 300 to be removed from the vessel after fistula formation while minimising damage to the vessel wall by the electrode 130.

The result of the fistula formation and valve destruction process according to FIGS. 6A-C is the same as that depicted in FIG. 3D.

Various modifications will be apparent to those skilled in the art.

The cutting unit 150 may not have a radially contracted configuration and a radially expanded configuration. For example, the cutting unit 150 may only have one configuration which allows it to cut the valve tissue.

The cutting tools 151a, 151b, 351a, 351b are not limited to a convex shape, but may be any other type of suitable shape, for example, a rectangular shape, a trapezoidal shape or triangular shape.

The cutting tools 151a, 151b, 351a, 351b may not have any spikes.

The cutting tools 151a, 151b, 351a, 351b may not have any recessed portions.

The electrode 130 is not limited to a ribbon wire, but may be any other type of suitable wire, for example, a cylindrical wire or oval wire.

The backstop 230 of the second catheter 200 is not limited to a concave shape but may also be any suitable shape. For example, the backstop 230 may be recessed or protruding and could have a concave, convex or rectangular shape.

The electrode 130 is not limited to a convex shape, but may be any other type of suitable shape, for example, a rectangular shape, a trapezoidal shape or triangular shape.

The electrode 130 is not limited to having a distal portion that is fixed to the housing and a proximal portion that is moveable. Alternatively, the proximal portion of the electrode 130 may be fixed to the housing 120 and the distal portion may be moveable. The electrode may be moveable between its expanded and contracted configurations by moving the sheath 160.

The cutting unit 150 or 350 is not limited to having cutting tools wherein the distal end of each cutting tool is fixed to the catheter body and the proximal end of each cutting tool is moveable within the catheter body. Alternatively, the proximal end of each of the cutting tools may be fixed to the catheter body 110 whilst the distal end of each of the cutting tools is moveable. The cutting unit may be moveable between its expanded and contracted configurations by moving the sheath 160.

The electrode expansion mechanism 410 is not limited to a slider 411, but may comprise any suitable mechanism which can move the electrode 130 between the radially expanded configuration and the radially contracted configuration. For example, the electrode expansion mechanism may comprise a linear actuator such as a rack and pinion.

The cutting unit control mechanism 420 is not limited to a slider 421, but may comprise any suitable mechanism which can move the cutting unit 150 or 350 between the radially expanded configuration and the radially contracted configuration. For example, the cutting unit control mechanism 420 may comprise a linear actuator such as a rack and pinion.

The handle 400 may comprise separate sliders for each cutting tool to control the expansion of each cutting tool individually.

The sheath sliding mechanism is not be limited to being included as part of the handle 400. Instead, the sheath sliding mechanism may be separate to the handle 400.

The sheath sliding mechanism is not limited to a slider, but may comprise any suitable mechanism which can move the sheath 160 longitudinally. For example, the sheath sliding mechanism may comprise a linear actuator such as a rack and pinion.

The cutting unit 150 or 350 is not limited to nitinol, but may be made from any suitable material such as stainless steel, for example.

The connecting elements 157a, 157b are not limited to nitinol, but may be made from any suitable material such as stainless steel, for example.

The connecting element 131 is not limited to the same material as the electrode, but may be made from any suitable material that is electrically conductive and resistant to heat, such as copper, iron or steel, for example.

The housing 120 of the catheter 100, 300 is not limited to a non-conductive ceramic material and may be made from any suitable material which can withstand the heat and plasma generated by the electrode 130. For example, the housing 120 may be made from a polymer such as polyimide.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A catheter for forming a fistula between two vessels, comprising:
a catheter body having a longitudinal axis;
an electrode extending radially from the catheter body for contacting a vessel wall and forming the fistula; and
a cutting unit disposed proximally or distally of the electrode for cutting a venous valve.

2. The catheter of claim 1, wherein the cutting unit is expandable and/or wherein the cutting unit has a radially contracted configuration and a radially expanded configuration.

3. (canceled)

4. The catheter of claim 1, wherein the cutting unit comprises a plurality of cutting tools.

5. The catheter of claim 4, wherein the cutting tools are arranged circumferentially around the catheter body, and wherein each of the cutting tools extend radially from the catheter body.

6. (canceled)

7. The catheter of claim 4, wherein each of the cutting tools has a substantially convex shape relative to the catheter body.

8. (canceled)

9. (canceled)

10. The catheter of claim 4, wherein the cutting tools extend longitudinally along the catheter body.

11. The catheter of claim 10, wherein each of the cutting tools has a proximal end and a distal end, and wherein the proximal end and distal end of each of the cutting tool is connected to the catheter body and wherein at least one cutting edge is disposed between the proximal end and distal end of each cutting tool.

12. (canceled)

13. (canceled)

14. The catheter of claim 13, wherein each of the cutting tools further comprises a spike positioned adjacent the cutting edge.

15. (canceled)

16. The catheter of claim 15, wherein the cutting edge is positioned in a recessed portion of the cutting tool.

17. The catheter of claim 4, wherein each of the cutting tools has a proximal section and a distal section.

18. The catheter of claim 17, wherein the cutting unit is disposed proximally of the electrode, and wherein the at least one cutting edge is disposed in the proximal section of each cutting tool and wherein the at least one cutting edge is facing proximally.

19. (canceled)

20. (canceled)

21. The catheter of claim 17 wherein the cutting unit is disposed distally of the electrode and wherein the at least one cutting edge is disposed in the distal section of each cutting tool and wherein the at least one cutting edge is facing distally.

22. (canceled)

23. (canceled)

24. The catheter of claim 18, wherein the distal end of each cutting tool is fixed to the catheter body and the proximal end of each cutting tool is moveable to move the cutting unit between a radially contracted configuration and a radially expanded configuration.

25. (canceled)

26. (canceled)

27. (canceled)

28. (canceled)

29. (canceled)

30. (canceled)

31. (canceled)

32. (canceled)

33. (canceled)

34. (canceled)

35. A system for forming a fistula between two vessels comprising:
a first catheter according to claim 1, and a second catheter comprising a second housing and a backstop for the electrode.

36. (canceled)

37. (canceled)

38. (canceled)

39. (canceled)

40. (canceled)

41. The system of any of claim 35, further comprising a sheath that is disposed over at least a portion of the catheter body and is slidably moveable along the longitudinal axis of the catheter body, and wherein the sheath is slidable to move the cutting unit between a radially contracted configuration and a radially expanded configuration and wherein the sheath is slideable to move the electrode between a radially contracted configuration and radially expanded configuration.

42. (canceled)

43. (canceled)

44. The system of claim 35, further comprising a handle disposed at a proximal end of the first catheter.

45. The system of claim 44, wherein the handle comprises a sheath sliding mechanism.

46. (canceled)

47. (canceled)

48. The system of claim 44, wherein the handle further comprises a cutting unit control mechanism for controlling the extent of radial expansion of the cutting unit and wherein the cutting unit control mechanism comprises a push wire that is connected to the proximal end of the cutting tools.

49. (canceled)

50. The system of claim 44, wherein the handle further comprises an electrode expansion mechanism for controlling the extent of radial expansion of the electrode and wherein the electrode expansion mechanism comprises a push wire that is connected to a proximal end of the electrode.

51. (canceled)

52. A method of forming a fistula using a catheter having a catheter body with an electrode and a cutting unit disposed proximally or distally of the electrode, the method comprising:
inserting the catheter into a vein through an access site;
moving the electrode from a radially contracted configuration to a radially expanded configuration;
forming the fistula by supplying RF energy to the electrode;
moving the cutting unit from a radially contracted configuration to a radially expanded configuration; and
cutting a valve by moving the catheter longitudinally in a proximal or distal direction.
